(19) United States
(12) Patent Application Publication
(10) Pub. No.: US 2007/0282316 A1
(43) Pub. Date: Dec. 6, 2007

(54) Method of prophylactically treating an artery to make it resistant to the subsequent development of atherosclerosis

(75) Inventors: Willard W. Hennemann, Hudson (CA); Daniel Nahon, Ottawa (CA)

Correspondence Address: John Christopher;Christopher & Weisberg, P.A., Suite 2040, 200 East Las Olas Boulevard, Fort Lauderdale, FL, 33301, US

(73) Assignee: CryoCath Technologies Inc.

(21) Appl. No.: 11/446,948

(22) Filed: Jun. 5, 2006

(51) Int. Cl. A61B 18/18

(52) U.S. Cl. 606/21

(57) ABSTRACT

The present invention provides a method for prophylactically treating a vessel region at risk for the development of vulnerable plaque with cryogenic energy. In general, a cryogenic catheter is inserted into the patient's vascular network and manipulated towards a treatment site. The catheter is then activated so as to cool the tissue at the treatment site to a predetermined temperature for a desired amount of time in order to induce the formation of scar tissue, which may include collagen or smooth muscle cell formation. It is understood that a variety of cryogenic catheter configurations can be used to cool the treatment site.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates generally towards treatment of vascular plaque, and more specifically to inhibiting the formation, progression, and/or rupture of an unstable (vulnerable) vascular plaque formation.

### BACKGROUND OF THE INVENTION

Coronary artery disease generally involves the formation of plaque, a combination of cholesterol and cellular waste products that form on the interior wall of an artery. Although the trigger that stimulates plaque formation is not completely understood, the first step in the process appears to involve dysfunction of the endothelial cell layer that lines the arterial wall. Lipids deposit on the surface and are absorbed into the artery wall. The increased lipids and locus of dysfunction leads to a release of proteins, called cytokines, that attract to inflammatory cells, called monocytes. The monocytes squeeze into the artery wall. Once inside the artery wall, the monocytes turn into cells called macrophages and begin scavenging or soaking up the lipids. The lipid-filled macrophages become foam cells, forming a plaque just under the surface of the arterial wall, often with a thin covering called a fibrous cap. The cytokines and the cascade of cellular and biochemical events may contribute to continued endothelial dysfunction, causing blood cells, mostly platelets, to begin to stick to the normally repellent vascular wall.

With plaque progression, the inflammation just under the surface erode the fibrous cap and can cause the plaque cap to crack, allowing the underlying plaque elements to come in contact with the blood stream. These underlying elements of lipids and collagen are highly thrombogenic. Exposure of these elements to the blood stream can cause clot formation, leading to coronary artery occlusion, myocardial ischemia and infarction. This particular type of lipid-rich plaque, having active inflammation and the potential to erode the overlying fibrous cap, which in turn can lead to thrombosis and myocardial infarction is called unstable or vulnerable plaque.

The current theory is that the underlying cause of most heart attacks is the development and rupture of these soft, unstable, atherosclerotic (or vulnerable) plaques in the coronary arteries. While the build up of hard plaque may produce severe obstruction in the coronary arteries and cause angina, it is the rupture of unstable, non-occlusive, vulnerable plaques that cause the vast majority of heart attacks.

Although vulnerable plaques may be detected, an ideal treatment for effectively treating these plaques does not exist. For example, treatments such as balloon angioplasty and/or stent therapy have been proposed for treating vulnerable plaques. However, many plaque lesions do not occlude the artery 60% or more and are therefore considered non-flow-limiting. The use of a balloon and/or stent in these situations can have the adverse effect of stimulating restenosis, thereby facilitating new clinical problems.

It is desirable, therefore, to provide a technique which may prevent the development of such plaque formations while not unnecessarily facilitating restenosis, and which may further stabilize or passivate plaque, thereby reducing the risk of plaque rupture.

### SUMMARY OF THE INVENTION

The present invention advantageously provides a method and system for prophylactically cryotreating a vessel at risk for the development of vulnerable plaque. For example, it has been shown that a large portion of heart attacks result from stenoses that originate in a proximal segment of the left anterior descending and proximal to mid segments of the right coronary artery. Due to such propensity for the development and subsequent rupture of plaque formations in that particular region, prophylactic treatment may be desired, without the need to first determine a specific location of an existing plaque deposit. Moreover, additional vascular regions may be identified as at-risk for plaque development through an analysis of an individual medical history of a patient, including factors known to increase the risk of plaque formation or development of coronary disease, such as diabetics, vessel disease, high levels of inflammation, or the like.

In a method of preventing subsequent development of atherosclerosis or plaque formations in human blood vessels, a cooling device may be positioned at an interior lumenal surface of a vessel at a point identified as being at risk for the development a plaque formation. The lumenal surface of the vessel is cooled to inhibit the development of plaque formation, where the lumenal surface is cooled to a low temperature for a time sufficient to cause the vessel wall to form a scar. The resulting scar may be resistant to the subsequent development or formation of atheroma or vulnerable plaque, and may include the formation of collagen or smooth muscle cells in the treated region.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates a surgical system for use with the method of the present invention;

FIG. 2 depicts an expandable thermally-transmissive region for use with the method of the present invention

FIG. 3 depicts an embodiment of a thermally-transmissive region for use with the method of the present invention;

FIG. 4 shows an alternative thermally-transmissive region for use with the method of the present invention;

FIG. 5 illustrates another thermally-transmissive region for use with the method of the present invention;

FIG. 6 shows an additional thermally-transmissive region for use with the method of the present invention;

FIG. 7 illustrates a method of use of an expandable thermally-transmissive region in accordance with the present invention; and

FIG. 8 illustrates a method of use of a thermally-transmissive region in accordance with the present invention.

### DETAILED DESCRIPTION OF THE INVENTION

The present invention provides a method for prophylactically treating a vessel region at risk for the development of vulnerable plaque with cryogenic energy. In general, a catheter is inserted into the patient's vascular network and manipulated towards a treatment site. The catheter is then activated so as to cool the tissue at the treatment site to a predetermined temperature for a desired amount of time in order to induce the formation of scar tissue, which may include collagen or smooth muscle cell formation. It is understood that a variety of cryogenic catheter configurations can be used to cool the treatment site.

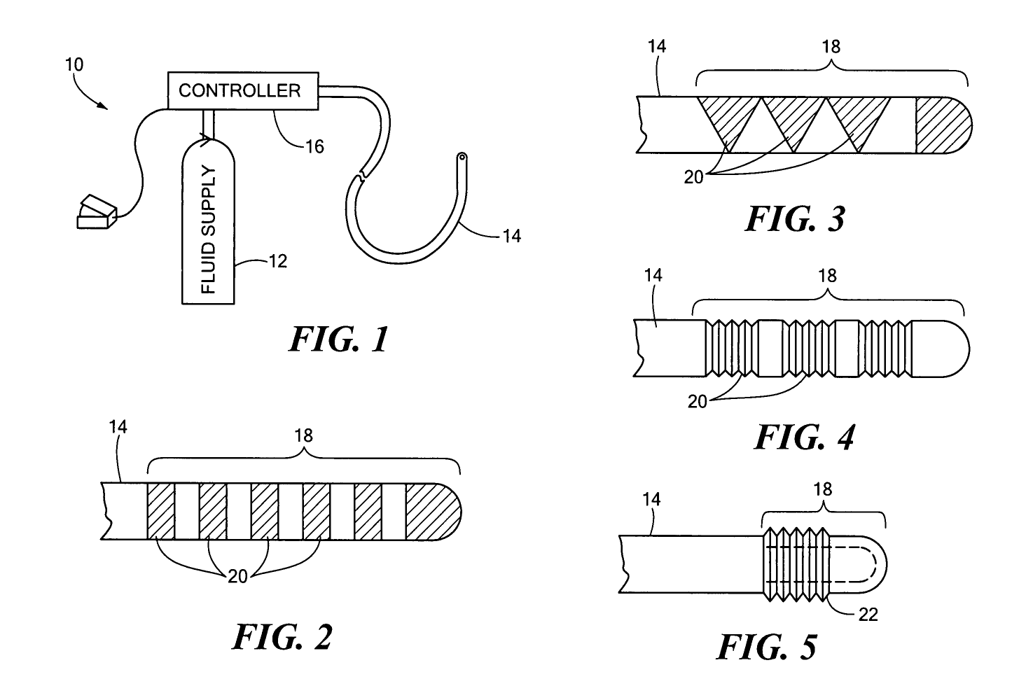

Referring now to the drawing figures in which like reference designators refer to like elements, there is shown in FIG. 1 a schematic illustration of an exemplary cryosurgical system 10 for use with the method of the present invention. The system includes a supply 12 of cryogenic or cooling fluid in communication with the proximal end of a flexible catheter 14. A fluid controller 16 is interposed or in-line between the cryogenic fluid supply 12 and the catheter 14 for regulating the flow of cryogenic fluid into the catheter 14 in response to a controller command. Controller commands can include programmed instructions, sensor signals, and manual user input. For example, the fluid controller 16 can be programmed or configured to increase and decrease the pressure of the fluid by predetermined pressure increments over predetermined time intervals.

One or more temperature sensors (not shown) in electrical communication with the controller can be provided to regulate or terminate the flow of cryogenic fluid into the catheter 14 when a predetermined temperature at a selected point or points on or within the catheter is/are obtained. For example, a temperature sensor can be placed at a point proximate the distal end of the catheter and other temperature sensors can be placed at spaced intervals between the distal end of the catheter and another point that is between the distal end and the proximal end.

The catheter may include a flexible member having a thermally-transmissive region 18 and a fluid path through the flexible member to the thermally-transmissive region 18. A fluid path is also provided from the thermally-transmissive region 18 to a point external to the catheter, such as the proximal end. Exemplary fluid paths include one or more channels defined by the flexible member, and/or by one or more additional flexible members that are internal to the first flexible member. In addition, the catheter may include a guidewire lumen or similar structure to provide for over-the-wire use of the device. Also, even though many materials and structures can be thermally conductive or thermally transmissive if chilled to a very low temperature and/or cold soaked, as used herein, a “thermally-transmissive region” is intended to broadly encompass any structure or region of the catheter that readily conducts thermal energy.

Now referring to FIG. 2, if it is desirable to treat an occluded region, a balloon 24 can be incorporated into the thermally transmissive region 18 such that the catheter 14 can dilate the occluded region of the vessel as well as treat the dilated region with cryogenic energy. Moreover, the catheter 14 may include one or more balloons or other expandable elements disposed about each other, or may alternatively include a multi-layered balloon structure.

Furthermore, while the thermally-transmissive region 18 can include a single, continuous, and uninterrupted surface or structure, it can also include multiple, discrete, thermally-transmissive structures that collectively define a thermally-transmissive region that is elongate or linear. For example, as shown in FIGS. 3 and 4, the catheter 14, or portions thereof, may have two or more thermally-transmissive segments 20 in a spaced-apart relationship. Each of the illustrated catheters includes a closed tip that can include a thermally-transmissive material. Depending on the ability of the cryogenic system, or portions thereof, to handle given thermal loads, the cooling of an elongate tissue path can be performed in a single or multiple cycle process without having to relocate the catheter one or more times or drag it across tissue.

In some embodiments, the thermally-transmissive region 18 of the catheter 14 may be deformable. An exemplary deformation is from a linear configuration to an arcuate configuration and is accomplished using mechanical and/or electrical devices known to those skilled in the art. For example, a wall portion of the flexible member can include a metal braid to make the catheter torquable for overall catheter steering and placement. Additionally, a cord, wire or cable can be incorporated with, or inserted into, the catheter for deformation of the thermally transmissive region.

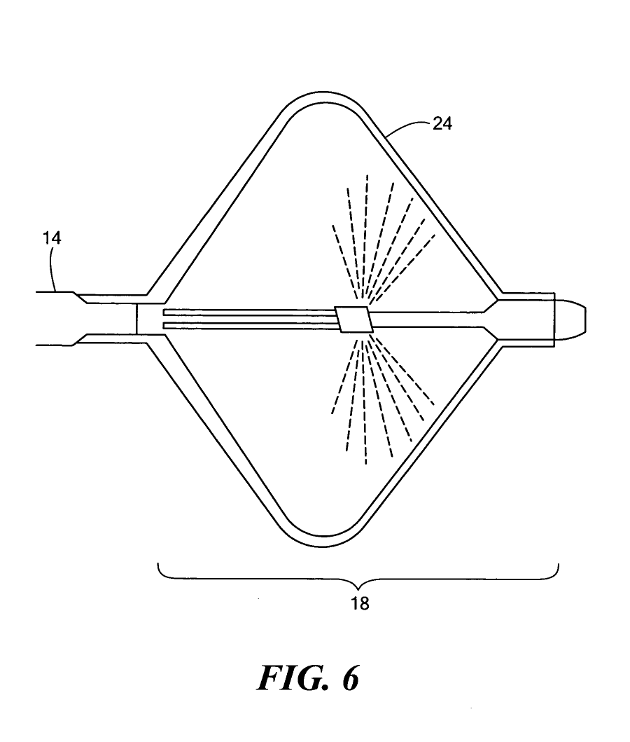

With respect to the embodiments shown in both FIGS. 3 and 4, the thermally-transmissive elements 20 are substantially rigid and are separated and/or joined by a flexible material. However, in other embodiments the thermally-transmissive elements 20 are flexible and are interdigitated with either rigid or flexible segments. FIG. 5, for example, illustrates an embodiment of the cryogenic catheter having three thermally-transmissive elements 20 that are flexible. The flexibility is provided by a folded or bellows-like structure. In addition to being shapeable, a metal bellows can have enough stiffness to retain a selected shape after a deforming or bending step. Instead of, or in addition to, flexible, thermally-transmissive elements and/or flexible material between elements, the distal tip (or a portion thereof) can be deformable. For example, FIG. 6 illustrates a tip having a thermally-transmissive, flexible bellows portion 22. It is understood that other types of cryogenic catheters having differing types of distal tips can be used to achieve the desired cooling of the target tissue region.

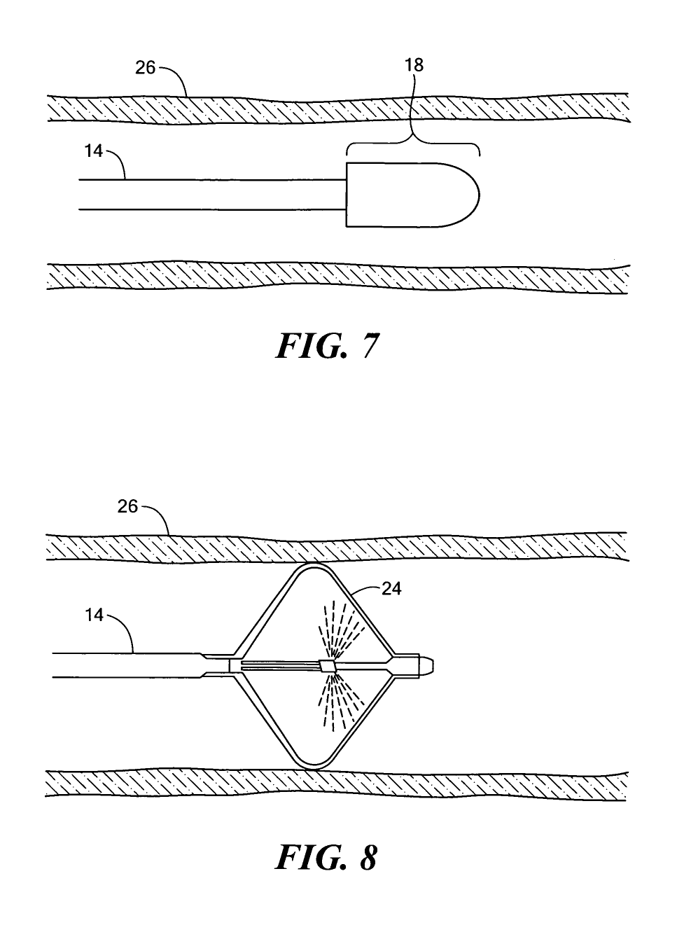

In an exemplary procedure, as shown in FIGS. 7 and 8, a thermally transmissive region 18 of a cooling device such as a catheter 14, which carries cooling fluid, is positioned in a vessel (body lumen) 26 at a region at risk for plaque development and/or formation on an interior lumenal surface. The particular region of tissue to be treated need not be identified as having an existing plaque formation, but rather may be identified as being at-risk to coronary disease or having an increased likelihood for subsequent plaque formation. Such risk-assessment and tissue identification may result from recognition of enhanced-risk circumstances, including vessel disease diabetics, elevated CRP levels and the like. By way of example, a patient may have differing risks of having a second coronary event at 12 months following a first event depending on whether the patient has single, double, or triple vessel disease. Patients with multiple risk factors (for example elevated CRP, triple vessel disease and diabetic) may be especially good candidates for prophylactic treatment since their likelihood of having a coronary event are very high. In addition, medical imaging instruments may be employed to identify patients or regions at risk for the development of plaque, including intravascular MRI, thermography, near infra-red spectroscopy, optical coherence tomography as well as non-invasive imaging such as CT and MRI. Further, it has been shown that a substantial percentage of heart attacks originate from plaque formation in the proximal 40-60 mm segment of the left anterior descending (LAD) and proximal to mid segments of the right coronary artery (RCA). As such, it may be highly desirable to treat these particular tissue regions, as well as tissue regions in the left circumflex artery (LCX).

Once positioned, the tissue of the surrounding vessel wall is cooled by a cryogenic process to a desired temperature and for a time sufficient to inhibit the metabolic and/or disease processes responsible for the formation and progression of plaque and/or to induce the formation of scar tissue.

In the embodiment shown in FIG. 7, a balloon catheter 14 may be positioned to contact and/or dilate a vessel region, and the balloon catheter 14 may be infused with a coolant and maintained in contact with tissue for a period of time as described above. A balloon catheter 14 is useful in situations where occlusion reduction is necessary and/or where a large area is being treated. In the latter case, the large contact area provided between the outer balloon surface and the vascular wall inner surface makes thermal energy transfer more efficient.

Irrespective of the particular device structure employed, the treatment site can be chilled in a wide range of temperatures and for various time intervals depending on the desired effect. For example, the tissue temperature can be held constant or it can vary. Further, the tissue can be chilled for one or more predetermined time intervals at the same or different temperatures. The time intervals can vary as well, so as to achieve a desired level of treatment for the target tissue. Also, certain areas of the treatment site may be cooled to a greater or lesser extent than surrounding target tissue.

During the cooling process as discussed above, a refrigerant such as nitrous oxide may be delivered under pressure such that expansion of the refrigerant occurs at a location within the catheter that is proximate to the target site, thereby cooling the tissue at and in the area near the target site. For example, treatment temperatures ranging from about ten degrees Celsius to about minus one hundred and twenty degrees Celsius, and preferably about zero degrees Celsius to about minus fifty degrees Celsius. The treatment may be applied for a duration lasting between approximately one second to about ten minutes.

In contrast with heat and radiation tissue treatments, cooling produces less damage to the arterial wall structure. The damage reduction occurs because a freeze injury does not significantly alter the tissue matrix structure as compared with the application of heat. Further, a freeze injury does not significantly reduce the reproductive/repair capability of the living tissue as compared with radiation treatments.

Positioning a catheter 14 inside the vascular vessel (i.e., the body lumen) 26, at approximately the point of the potential vulnerable plaque development, and cryogenically treating the region may advantageously arrest the metabolic process and/or disease responsible for the instability, as well as increase the thickness of the vessel wall by stimulating collagen synthesis and/or smooth muscle cell growth. The result may include the creation of a scar or other tissue formation which may significantly reduce the likelihood of subsequent plaque formation. It has been shown that a freeze injury will increase the level of collagen matrix within the treated segment. By applying such a cryogenic treatment to the vulnerable plaque that is at high risk of rupture, the plaque may be stabilized by increasing its collagen content and creating scar tissue that will make it less likely to rupture

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of preventing development of atherosclerosis in a vessel, comprising the steps of:
positioning a treatment element having a thermally-transmissive region into a lumen of the vessel;
cooling the vessel to a predetermined temperature for a predetermined time duration.

2. The method according to claim 1, further comprising the step of forming a scar, wherein the scar is resistant to the development of atheroma or vulnerable plaque.

3. The method according to claim 1 wherein the step of cooling increases the level of collagen in the vessel.

4. The method according to claim 2, wherein the step of cooling reduces the level of hyperplastic smooth muscle cell proliferation.

5. The method according to claim 2, wherein the step of cooling increases the level of hyperplastic smooth muscle cell proliferation.

6. The method according to claim 1, wherein the step of cooling the vessel includes circulating a cryogenic fluid through the treatment element.

7. The method according to claim 6, wherein the vessel is cooled to a temperature between 0° C. and −30° C.

8. The method according to claim 7, wherein the vessel is cooled for a duration of between 1 second and 180 seconds.

9. The method according to claim 1, further comprising the step of identifying a patient at risk for development of vulnerable plaque.

10. The method according to claim 9, wherein the step of identifying a patient at risk includes identification of at least one risk factor.

11. The method according to claim 10, wherein the at least one risk factor is one of diabetic condition, recent coronary syndrome, and increased tissue inflammation level.

12. The method according to claim 1, wherein the vessel is one of the left anterior descending segment and proximal to mid segments of the right coronary artery.

13. The method according to claim 1, wherein the vessel is the left circumflex artery.

14. A method of prophylactically treating a vessel prior to the development of vulnerable plaque, comprising the steps of:
positioning a treatment element having a thermally-transmissive region into a lumen of the vessel;
cooling the vessel to a temperature between 10° C. and −100° C. for a duration between 1 second and 180 seconds to form a scar, wherein the scar is resistant to the development of atheroma or vulnerable plaque.

15. The method according to claim 14, wherein the step of cooling increases the level of collagen in the vessel.

16. The method according to claim 14, wherein the step of cooling reduces the level of hyperplastic smooth muscle cell proliferation.

17. The method according to claim 14, further comprising the step of identifying a patient at risk for development of vulnerable plaque.

18. The method according to claim 17, wherein the step of identifying a patient at risk includes identifying at least one risk factor.

19. The method according to claim 14, wherein the vessel is one of the left anterior descending segment and proximal to mid segments of the right coronary artery.

20. The method according to claim 14, wherein the vessel is the left circumflex artery.

21. A method of prophylactically treating a vessel prior to the development of vulnerable plaque, comprising the steps of:
positioning a treatment element having a thermally-transmissive region into the left anterior descending right coronary artery;
cooling the segment to form a scar, wherein the scar is resistant to the development of atheroma or vulnerable plaque;
positioning a treatment element having a thermally-transmissive region into the proximal to mid segments of the right coronary artery;
cooling the segment to form a scar, wherein the scar is resistant to the development of atheroma or vulnerable plaque;
positioning a treatment element having a thermally-transmissive region into the a segment of the left circumflex artery; and
cooling the segment to form a scar, wherein the scar is resistant to the development of atheroma or vulnerable plaque.
